(19) United States
(12) Patent Application Publication
(10) Pub. No.: US 2020/0337583 A1
(43) Pub. Date: Oct. 29, 2020

(54) HEART MONITORING SYSTEM AND METHOD OF USE

(71) Applicant: Bernard S. Ku, II, Austin, TX (US)

(72) Inventor: Bernard S. Ku, II, Austin, TX (US)

(21) Appl. No.: 16/039,839

(22) Filed: Jul. 19, 2018

(51) Int. Cl. A61B 5/0408; A61B 5/024; A61B 5/00; A61B 5/0456

(52) U.S. Cl. CPC A61B 5/04087; A61B 5/02438; A61B 90/92; A61B 5/684; A61B 5/0456; A61B 5/04085

(57) ABSTRACT

A heart monitoring system enables a person or user to create and evaluate and ECG without the assistance of a medical professional. A self-adhesive patch with leads is placed over the heart of the person and places the leads in an optimal position to thoroughly monitor the patterns emitted from the heart. The data samples are transmitted to a personal device for display and analysis.

### BACKGROUND

### 1. Field of the Invention

The present invention relates generally to healthcare systems, and more specifically, to systems for monitoring and recording the electrical patterns of the heart.

### 2. Description of Related Art

Healthcare systems are well known in the art and are effective means to alleviate disease or improve function of a body. For example, FIG. 1 depicts a conventional portable electrocardiogram (ECG) system 101 having a band 103 with two leads 105a and 105b imbedded within it. The band 103 transmitting data to a CPU 107. During use, the band 103 holds the leads 105a and 105b against the skin so that the leads 105a and 105b capture the heart rhythms of the person wearing the band. The data taken from the leads 105a and 105b is transmitted to the CPU 107 to be displayed to the person or to be recorded.

One of the problems commonly associated with system 101 is its limited use. For example, the use of two leads limits what can be captured and reported resulting in missed signs of heart problems.

Commonly available portable ECG systems do not collect repetitive measurements to establish a recent history of the heart over a recent time period. This baseline represents a normalized ECG and allows for the elimination of normal deviations in the hearts performance when considering the overall health of the heart.

Additionally, the baseline of the person is extremely important when evaluating the current measurements taken by the leads however with the limited measurement capabilities of a two lead system combined with the simple display do not enable proper evaluation of the heart.

In comparison medical professionals in hospitals or clinics use twelve (12) leads, precisely located in the area of the torso to monitor the heart and establish a baseline for the person to evaluate the condition of the heart.

Additionally, current ECGs collected in hospitals or clinics are stored in the specified location where they were obtained and are not shared among other hospitals, clinics or professionals. Creating a potential health hazard when the patient travels away from their home and their ECG results are not immediately available for comparison or diagnosis.

The problems with the current portable ECG systems are further complicated by the lack retaining measurements over time. The low quality of these portable systems and the limited display renders the data useless and so are not stored for future use or comparison.

The ECG is used to detect many conditions of the heart such as, abnormally fast or slow heart rhythms, abnormal conduction of cardiac impulses, evidence of prior or evolving heart attacks, acute impairment to the blood flow, electrolyte levels, inflammation and congenital heart conditions or other indicators to suggest structural heart disease. The indicators for many conditions require detection of minute measurements and comparison of these measurements to previous measurements.

The ECG is also used to monitor the effects of heart medications to allow for adapting medication strength to meet the needs of the patient. These effects could also be of a negative nature and would cause the cessation of the medication if identified. As these effects require current professional level measurements the patient must travel to a medical facility and consume the time of medical professionals at preregular intervals to ensure safe medication.

The health of the heart and the stress thereon are measured through an ECG. The heart is subjected to elevated demands in circulation and is monitored to see if the heart can meet these demands. Patients with pacemakers, defibrillators or other biotelemetric devices frequently need ECGs to monitor the state of the device and the heart affected thereby.

The ECG in its previously discussed uses is necessary to detect conditions in the heart prior to their causing serious problems that could include loss of life. Currently delayed access to trained medical or healthcare professionals particularly specialist cause patients to increase the interval between readings or they are omitted. Another cause of delay or neglect is the cost that is incurred for the testing.

When a person obtains professional grade ECG measurements care the results are generally kept by the medical professional or their organization. When the results are stored and used in the future for comparison of a new measurement the time lapse between the two is usually a month to year old in which time normal deviation and change in the heart reduces the certainty of the comparison. It would be beneficial to have recent results such as every day for a proceeding week with which to compare the current measurements. The difficulties discussed above limit the availability of obtaining these recent sequential measurements to only high-risk patients or others with suspected conditions requiring a high level of attention.

Accordingly, although great strides have been made in the area of portable ECG systems, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a front view of a common portable ECG system;

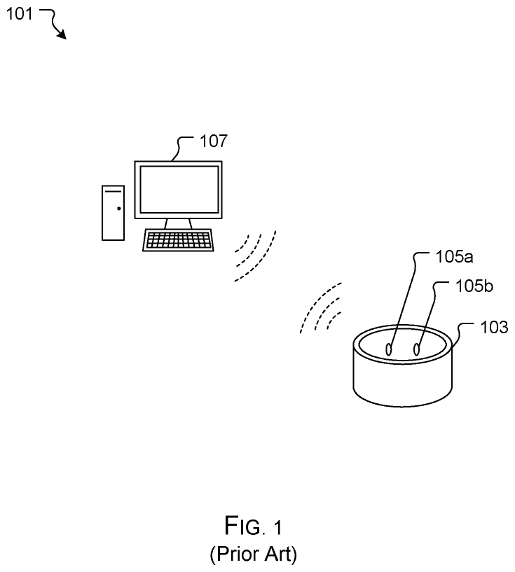

FIG. 2 is a front view of a heart monitoring system in accordance with a preferred embodiment of the present application; and

FIG. 3 is a back view of the self-adhesive patch of FIG. 2;

FIG. 4 is a diagram of the personal device of FIG. 2;

FIG. 5 is a diagram of the interface of FIG. 4;

FIG. 6 is a screen depiction of ECG data acquired with the system of FIG. 2;

FIG. 7 is a flow chart of the status reporting of the interface of FIG. 4;

FIG. 8 is a diagram of the reporting options of the interface of FIG. 4; and

FIG. 9 is a flow chart of the initialization of the interface of FIG. 2.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional portable ECG systems. Specifically, the invention of the present application enables a near professional measurement without the need to be administered by a professional or in a medical facility. In addition, the invention enables the creation and continuation of a baseline. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

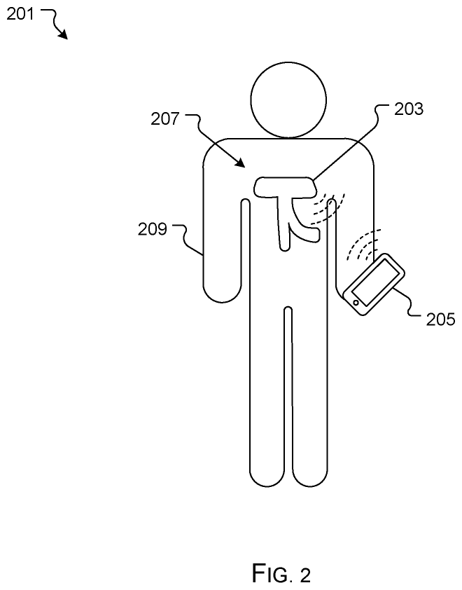

Referring now to the drawings wherein like reference characters identify corresponding or similar elements throughout the several views, FIG. 2 depicts a front view of a heart monitoring system in accordance with a preferred embodiment of the present application. It will be appreciated that system 201 overcomes one or more of the above-listed problems commonly associated with conventional portable ECG systems.

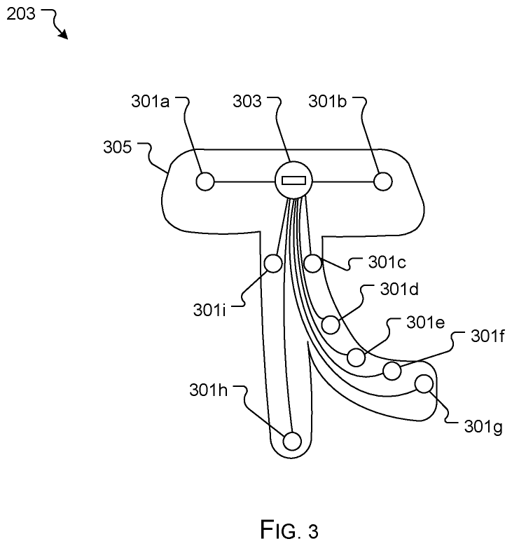

In the contemplated embodiment, system 201 includes a self-adhesive patch 203 in electronic communication with a personal device 205 such as a smart phone, tablet, computer or the like. As depicted by the FIG. 3, the self-adhesive patch 203 including nine leads 301 in electric communication with a first data port 303 attached to an adhesive body 305. It will be appreciated that the adhesive body is shaped so as to place the leads 301 in the locations where essential data about the heart is available.

In use, the self-adhesive patch 203 is placed on the torso 207 of a user 209 and capture data related to the user's heart.

In the current embodiment 201 the self-adhesive patch 203 is contemplated to be applied to the torso 207 by the user 209. This self-application eliminates the need to have the plurality of leads 301 attached to the user 209 by a medical professional greatly increasing the availability of taking high quality measurements and freeing the medical professionals to provide improved care to all their patients.

It is further contemplated that the self-adhesive patch 203 could vary is size and be altered to accommodate users 209 of various sized from children to adults.

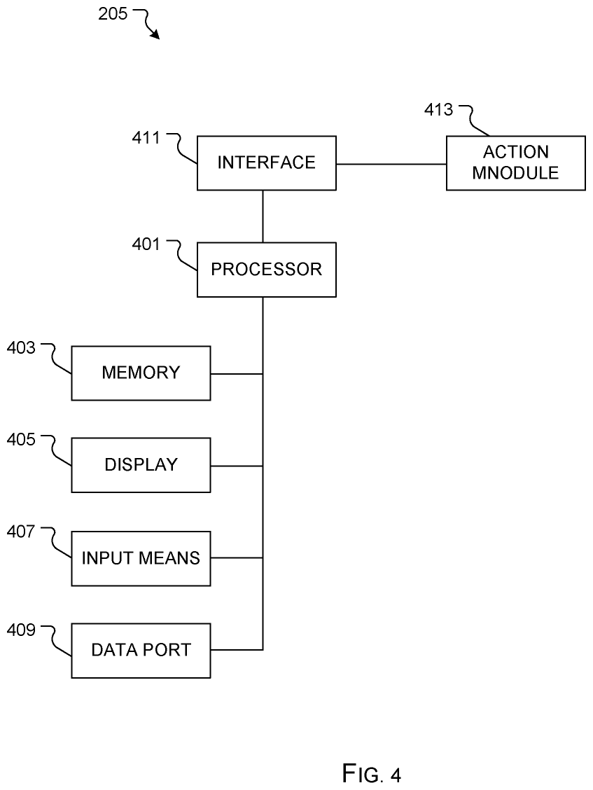

Referring now to FIG. 4 the personal device 205 is depicted including processor 401, memory 403, a display 405, an input receiver 407 and a second data port 409 all in communication and configured to enable an interface 411 to capture data from the self-adhesive patch 203 and use the data to establish a baseline. The memory 403 enables the retention of each measurement taken by the system 201. The interface also including an action module 413 containing tasks that should be taken when a key indicator is identified by the interface in the measurements of the user's heart.

It should be appreciated that one of the unique features believed characteristic of the present application is that the number of leads 301 included in the self-adhesive patch 203 capture an increased amount of important details in the performance of a user's heart. These details that obtained through having multiple leads 301 enable the detection of conditions in the heart that could lead to catastrophic events that would evade treatment without the increased number of leads 301 in the system 201.

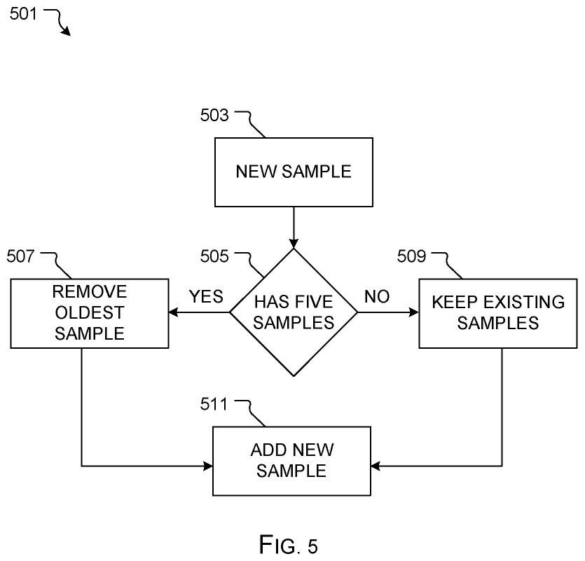

Referring to FIG. 5, in the preferred embodiment, interface 411 creates a running baseline 501 with data acquired from the five most subsequent uses. When a new sample 503 of data is received from the self-adhesive patch 203 the interface 411 determines if five samples are currently in the baseline 505. If five samples exist the oldest is removed 507 and if not the existing samples are retained 509. The new sample is then added to the baseline. While the number five is given it is an example only and any number is contemplated so as the baseline 501 is generated therefrom. It is also contemplated that all samples could be stored via the interface 411. While five samples are given here, any number of samples could be used to establish the baseline and not deviate from the intent of the invention described here.

Another unique feature believed characteristic of the present application is that interface 411 captures and stores measurements from the leads 301 and generates a current high-quality readout and baseline of the hearts status.

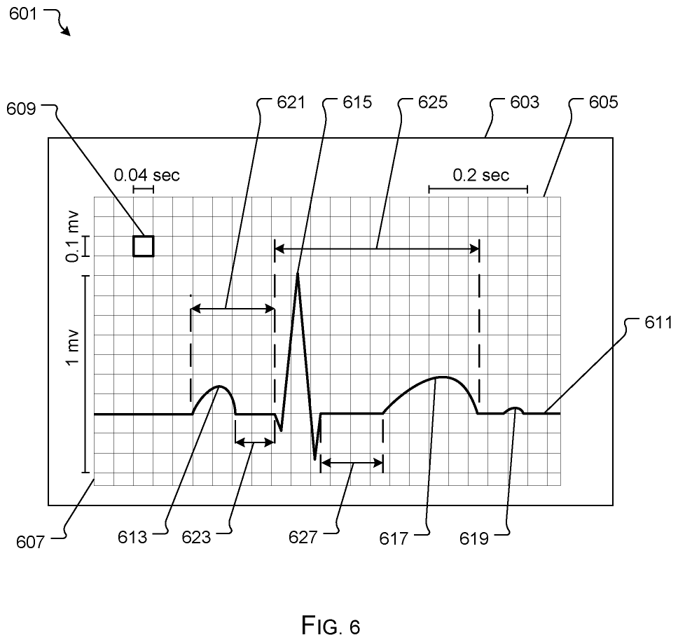

The system 201 is contemplated to generate the readout as depicted by FIG. 6. It is intended that the readout 601 is of professional quality and conformity to facilitate the comparison of the measurements taken by the heart monitoring system 201 and professionally taken measurements. It will be appreciated that this ability, to compare personal and professional measurements greatly increases the ability of medical professionals to identify and treat heart conditions that would otherwise go unnoticed.

It is contemplated that the readout 601 includes a grid 603 preferably having time in 0.04 second increments on a first axis 605 and voltage in 0.1 millivolts on a second axis 607. These axes 605, 607 forming grid boxes 609.

The sample data 611 being displayed on the grid 603. Attributes of the sample data 611 could include features such as a P wave 613, a QRS complex 615, a T wave 617, a U wave 619, a PR interval 621, a PR segment 623, a QT interval 625 or a ST segment 627. It will be understood that these features will vary in magnitude and duration and that the value of system 201 is the ability to first detect and then measure these variations.

The P wave 613 is a smooth mound looking hump that proceeds the QRS complex 615 and is measurement of atrial depolarization. Average amplitude is 0.15 my with a duration of 0.05-0.1 seconds. The P wave 613 could be normal, inverted, duplicated, wavy or not present and vary in amplitude and duration.

The QRS complex 615 has three components regularly, however, one could be missing, and appears as a spike following the P wave 613 and represents ventricular depolarization. The average duration is 0.1 seconds and could be normal, inverted, or vary in amplitude and duration.

The T wave 617 is a rounded bump that follows the QRS complex 615 and is a measurement of ventricle repolarization. The T wave 617 could be normal, multi-phasic, inverted and vary in amplitude and duration

The U wave 619 is a smaller round bump that follows the T wave 617 when present.

The PR interval 621 measures the delay in conduction of electrical impulses between the upper and lower heart chambers. This interval includes the time of the P wave 613 and averages 0.16 seconds.

The PR segment 623 is the activity of the heart between the end of the P wave 613 and the beginning of the QRS complex 615 and represents the time of the impulses going to the AV nodes (special electrical conduction states between the upper and lower chambers).

The QT interval 625 measures the total time of ventricular activity including the duration of the QRS complex 615 (depolarization) and T wave (repolarization). Average duration is 0.4 seconds. This interval will vary by patient dependent on age, gender and heart rate.

The ST segment marks the activity between the end of depolarization and the repolarization of the ventricles or the time between the end of the QRS complex 617 and the beginning of the T wave 617. During the T wave the ventricles begin to repolarize. The ST segment could be normal, concave, convex, elevated, depressed, slopped or vary in amplitude and duration.

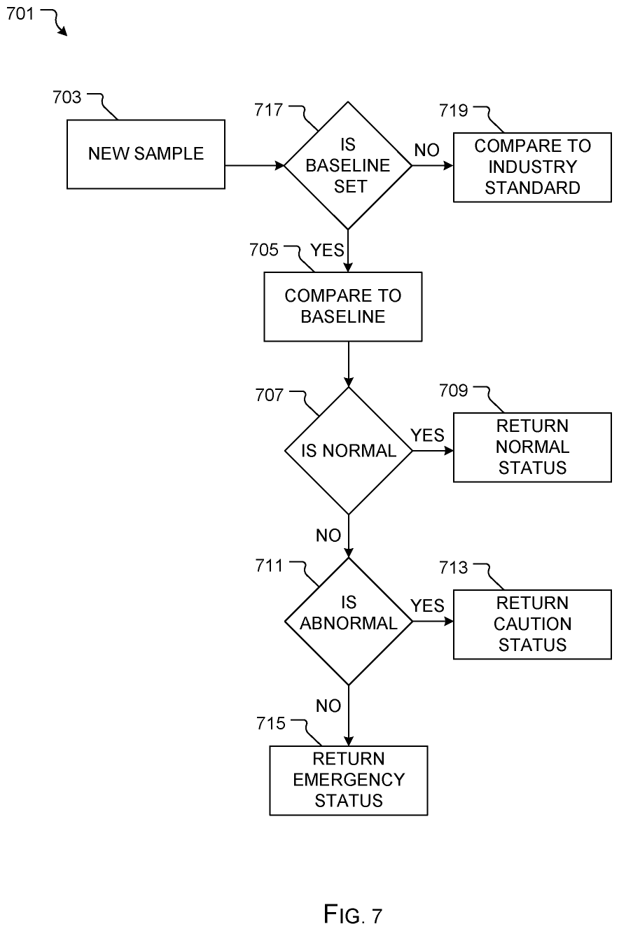

The interface 411 of the system 201 is contemplated to analyze the sample data 611 and compare the baseline 501 therewith to provide the user 209 with a status of their heart as depicted in FIG. 7. The contemplated embodiment 701 this analysis includes acquiring the new sample 703 and if a baseline is established 717 comparing it to the established baseline 705. If a baseline is not established the portal proceeds to select a most applicable industry standard to compare the new sample against 719.

It will be understood that industry standards use general population data to normalize and predict what an ECG for that person matching these characteristics should be. After comparing the new sample to the baseline if it is normal 707 returning a normal status to the user 709, if not then evaluating if it is abnormal 711 and if so returning a caution status 715, if the new sample deviates beyond a threshold set in the interface to define abnormal then returning an emergency status 715 as this amount of deviation is considered potentially dangerous, life threatening or requiring medical attention.

It is contemplated that information relevant to locating an industry standard is available to the interface 205 through a network or placement at the time of origination thereof.

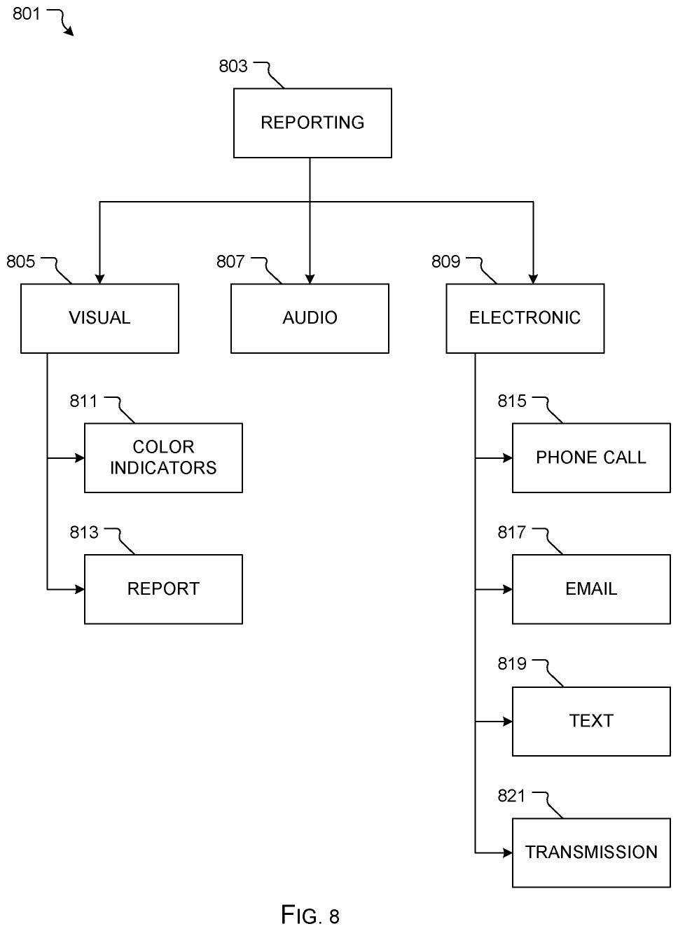

It is contemplated that the status return 801 as depicted by FIG. 8 includes a reporting module 803 that enables options for the user 209 to select from such as visual 805, audio 807 and electronic 809. It will be understood that these options are given as examples and not intended to limit the scope of the invention. It is further contemplated that the visual 805 options provide for color 811 changes to some part or portion of the displayed results such as green could stand for normal, yellow could be for abnormal and red could be for dangerously abnormal. A report 813 could also be displayed with raw, summarized or interpreted information being shown.

The report 813 is contemplated to be available as it is generated, after is stored or as a compilation of the baseline established by the interface 411. It is also contemplated that the condition and place of storage for single or multiple reports could be determined by the user. It will be appreciated that in this manner ECG data for the user could be on the user's person wherever they might be, including away from home.

It will be appreciated that the notification of the status of the measurements or their comparison to the baseline 501 greatly improves the ability of the user 209 to understand their state and could seek medical attention. In a similar manner the suggested notification of a medical professional of the same measurements or their comparison to the baseline 501 allows for prompt review and interpretation if desired. It will be understood that the ability of medical professionals and particularly specialists is not intended to be replaced by the heart monitoring system 201 but could be augmented thereby.

With the use of the personal device 205 in any setting and at the discretion of the user 209, it is contemplated that the electronic 809 options could allow for an email 817 or text message 819 to be sent to the user 209 themselves, a relative or a care provider with any combination of data or analytical results. It is contemplated and will be appreciated that if the results are considered potentially dangerous that a call 815 could be initiated to emergency response teams or another medical professional. It is also contemplated that a phone call 815 could be initiated in other situations such as to a nurse to review the results, provide positive feedback for taking the measurements or any other subject matter.

It is further contemplated and will be appreciated that another electronic option 809 could be the transmission 821 of the measurement data to a server, printer, fax machine or the like. It will be appreciated that in this option a physical copy or rendition of the measurements, baseline or other output from the interface is created. For example, the user could print a copy or request a printed copy, a doctor could receive and review, a clerk adds the data to a file for storage and so on. It will be appreciated that these options 809 are of value to professionals as well as during personal use.

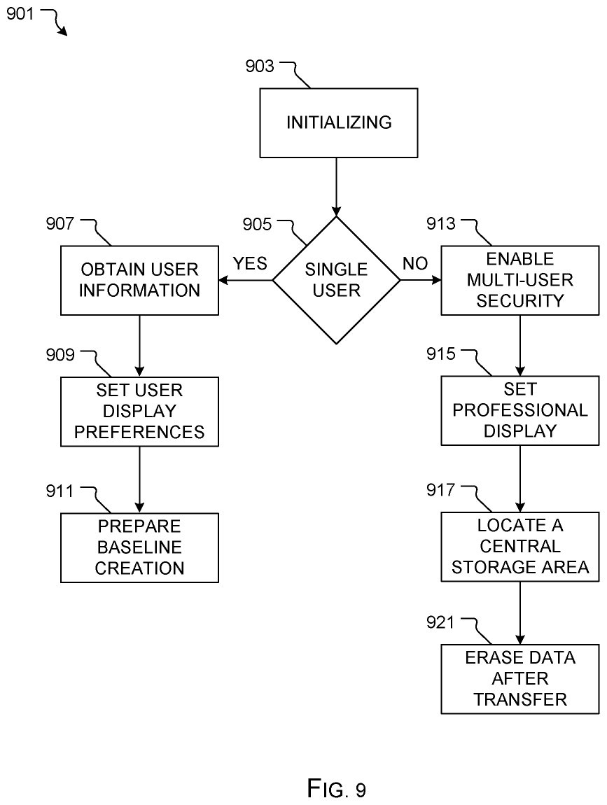

Referring now to FIG. 9, the use of the system 201 in both personal and professional areas is depicted. Embodiment 901 preparing the interface for use could require initialization 903 thereof. It is contemplated that the interface could be used for a single user 905 or multiple users as commonly found in professional medical practice. It is given as an example that for a single user the interface 901 could ask for personal information 907, preferences 909 and preparing a baseline 911 for the user. It is also contemplated that in a professional setting the security of each user 913 must be considered, that doctors, nurses, specialists and so forth could desire data to be displayed 915 in depth or that the system function on a set time frame or the like, that new samples acquired by the system could be sent to secure storage area 917 as they are taken and that no data remain in the system 921 after use. These are given as examples and are not intended to limit the scope of the invention. It is contemplated that other features or functions could be desired both by the self-user or the professional.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed:

1. A heart monitoring system comprising:
a self-adhesive patch having a plurality of leads attached thereto; and
the leads in communication with a first data port;
a personal device having a second data port and configured to support an interface;
wherein the first and second data port enable the data acquired from the plurality of leads to be transmitted to the personal device; and
wherein the interface enables the creation of a baseline defining the status of the heart.

2. The system of claim 1 wherein the number of leads attached to the self-adhesive patch is nine.

3. The patch of claim 1 wherein the patch comprises a single adhesive body.

4. The system of claim 1 wherein the patch is applied by the user to themselves or to another person.

5. The system of claim 1 wherein the use of the patch or interface is personal or professional.

6. The system of claim 1 wherein data from the leads are stored via the personal device or a machine-readable storage medium, comprising executable instructions that, when executed by a processing system including a processor, facilitate performance of operations, comprising: receiving a request from a communication device to initiate a communication session; responsive to determining that there is an potential health risk of the heart, obtaining a personal baseline, storing the personal baseline and storing subsequent ECG records in a database for future reference and processing.

7. A heart monitoring system comprising an interface configured to compare new data acquired via a plurality of leads to a baseline previously established from data acquired in a like manner.

8. The system of claim 7 wherein the baseline is established with at least one set of data acquired from the plurality of leads.

9. The system of claim 7 wherein the interface reports the results of the comparison.

10. The system of claim 9 wherein the results are reported by altering the color of some portion of the interface.

11. The system of claim 10 wherein the color is changed to green for a normal result, yellow for an abnormal result and red for a limit exceeded result.

12. The system of claim 7 wherein the interface provides reporting options to a user.

13. The system of claim 12 wherein the options include, visual, audio or electronic methods.

14. The system of claim 7 wherein the new data, the baseline or interpolated data are stored by the interface.

15. The system of claim 7 wherein the interface comprises an action module that provides recommended tasks to the user based on the results of the new data, baseline or interpolated data.

16. The system of claim 7 wherein the baseline is replaced by an industry standard respective to information about the user.

17. A heart monitoring system comprising an interface configured to acquire new data via a plurality of leads and stored therein wherein the interface is active on a personal device that is carried on the person of the user.

18. The system of claim 1 wherein the data stored on the personal device is configured to be retrieved and shared with at least one medical professional.

19. A heart monitoring system comprising a plurality of leads in communication with an interface wherein measurements of the heart are taken by the leads, transferred to the interface and recorded therein for interpolation with respect to an electrocardiogram.

20. The system of claim 17 wherein the data comprises a P wave, QRS complex, T wave, U wave, PR interval, PR segment, QT interval or ST segment.
